(19) United States
(12) Patent Application Publication
(10) Pub. No.: US 2014/0025151 A1
(43) Pub. Date: Jan. 23, 2014

(54) Retrievable stent for intracranial aneurysms

(71) Applicant: BULANG GAO, Lynn, MA (US)

(72) Inventor: BULANG GAO, Lynn, MA (US)

(21) Appl. No.: 13/553,883

(22) Filed: Jul. 20, 2012

(51) Int. Cl. A61F 2/82; A61F 2/84; A61F 2/86

(52) U.S. Cl. 623/1.11; 623/1.2; 623/1.34; 623/1.13; 623/1.19; 623/1.18

(57) ABSTRACT

A retrievable stent is a self-expandable or balloon-expandable stent which is very useful for treating intracranial sidewall aneurysms and can be retrieved especially when the stent is not deployed appropriately. The stent comprises a proximal retrieving structure and a distal self-expandable or balloon-expandable stent or covered stent for the treatment of sidewall aneurysms of cerebral blood vessels.

### FIELD OF THE INVENTION

The present invention relates to medical devices used to treat aneurysms within diseased blood vessels, and more particularly, relates to medical devices used to close the neck of intracranial sidewall aneurysms of cerebral blood vessels. Examples of blood vessels in which the said retrievable stent may be implanted include anterior cerebral artery, the middle cerebral artery, the internal carotid artery, and the basilar artery.

### BACKGROUND OF THE INVENTION

Although the following discussion focuses on the treatment of vascular diseases, it is equally applicable to diseases in other locations or tracts. Although many types of vascular diseases can be treated with this retrievable covered stent such as aneurysms, arteriovenous fistulas, vascular defects and stenosis, the following discussion focuses on the treatment of intracranial sidewall aneurysms which are one very significant use for the retrievable stent.

An aneurysm is a sac formed by localized dilatation of the wall of an vessel. Common areas where aneurysms occur and cause potential medical conditions include the coronary arteries, the carotid arteries, various cerebral arteries and the abdominal aorta. The wall of an aneurysm may progressively dilates, weakens and ruptures, causing dramatic negative health consequences such as a stroke or death when a cerebral aneurysm or an abdominal aortic aneurysm ruptures. Aneurysms can be treated surgically or endovascularly. The surgical procedure, however, is extremely traumatic and presents a high level of risk, particularly when treating cerebral aneurysms. To avoid the high risk of vascular surgery, endovascular devices have been used to either cover or fill an aneurysm with embolic materials such as coils. One method of endovascular treatment is to fill the aneurysm cavity with coils alone or with coils in combination with stents. When using coils in combination with stents (this technique is also called stent-assisted coil embolization), a non-covered stent has to be placed across the aneurysm neck in advance before coil embolization is performed through the stent struts into the aneurysm cavity. Once the aneurysm cavity is filled with coils, blood will not enter the aneurysm, and the aneurysm will be expelled from blood circulation. However, for large aneurysms, a lot of coils have to be used, thus creating a mass effect with severe consequences. Moreover, a lot of other severe complications may also occur with coiling embolization such as coil protrusion or escaping, intra-procedural aneurysm rupture leading to subarachnoid hemorrhage, and thrombosis caused by coils leading to cerebral embolism. These complications limit its application. Another infrequently-used method is the deployment of a covered stent across the aneurysm neck, and this approach is a much better method than embolization of the aneurysm with embolic materials such as coils alone or coils combined with stents. The covered stent's membrane or graft can act as a barricade to prevent blood from entering the aneurysm sac, leading to thrombosis within the sac, consequently expelling the aneurysm from blood circulation. At the same time, the parent artery harboring the aneurysm is protected. Since manipulation of the covered stent occurs within the parent artery rather than within the aneurysm cavity in case of coil emoblization, there is no possibility of intra-procedural aneurysmal rupture. Furthermore, the thrombus formed within the aneurysm sac will not be dislodged by the covered stent, which greatly decreases the possibility of thromboembolic complications. For patients with very large or giant aneurysms where embolization could require the placement of many costly coils which may result in a mass effect, the deployment of a covered stent over the aneurysm orifice may sufficiently occlude the aneurysm without causing those side effects mentioned above. Moreover, small and recurrent aneurysms difficult to treat can also be easily completely occluded by a covered stent.

An arteriovenous fistula (AVF) is an abnormal connection between an artery and a vein, and the carotid-cavernous fistula (CCF) is an abnormal shunt between the intracavernous internal carotid artery (ICA) and the cavernous sinus. They are usually caused by trauma, but a ruptured aneurysm may also cause a CCF. The goal of treatment of AVF is to eliminate the fistula with concurrent preservation of the vessel. Surgical therapy may or may not be applicable depending on the location of the fistula, and surgery may not be efficient for CCF. With continuous and rapid evolution of endovascular technology, an increasing number of AVFs have been successfully treated through endovascular means. The endovascular management of AVFs include transarterial and transvenous embolization involving treatment materials like detachable balloons, coils, NBCA, and absolute alcohol. The classic technique for the treatment of CCFs involves transarterial introduction of detachable balloons, coils and other embolic materials into the cavernous sinus to occlude the fistula with concurrent preservation of the ICA. However, the use of a covered stent may greatly simplify the treatment procedure and reduce the cost as well because the graft on the stent can occlude the fistula and provide a prosthetic lumen for the flow of blood.

Vascular stenosis frequently arises from atherosclerotic disease and even results in occlusion of the involved vessel, causing corresponding ischemic symptoms. The frequently affected vessels are ICAs, vertebral arteries, intracranial vessels, subclavicular arteries, coronary arteries, iliac arteries, arteries of the extremities and so on. Stenosis or even occlusion of coronary arteries, for example, is a common cause of heart attack. Diseased and obstructed coronary arteries can restrict the flow of blood in the heart and cause tissue ischemia and necrosis. Surgical construction of coronary artery bypass grafts is often the method of choice when there are several diseased segments in one or multiple arteries. Conventional open-heart surgery is, of course, very invasive and traumatic for patients undergoing such treatment. Therefore, alternative methods being less traumatic are highly desirable. One of the alternative methods is balloon angioplasty to open the stenotic artery, and another one is atherectomy, a technique in which occlusive atheromas are cut from the inner surface of the arteries. Both methods suffer from reocclusion with certain percentage of patients. Endovascularly, a bare metallic stent can be placed at the stenotic position to open and maintain the vessel. However, neointima may grow through the stent struts and cause restenosis of the artery. The deployment of a covered stent can effectively open the stenotic site of vessel, maintain the vessel in an open state, provide a fluid pathway in the previously stenotic and even occluded vessel, and maintain long-term patency with its membrane which can prevent the inner growth of the neointima.

Vascular defects can also be repaired with a covered stent which can provide a prosthetic lumen for the flow of blood or other liquid and is deployed to replace, supplement or exclude portions of blood vessel or bodily tracts. The membrane on the stent is a tubular graft secured onto the inner or outer diameter of the stent support structure and can maintain the integration of the bodily tract so that blood or other liquid can flow within the tubular graft of the covered stent, consequently reconstructing the defect of the bodily tract.

However, a frequently encountered problem in deploying a stent for treating the above diseases are mis-deployment of the stent. If the stent is deployed in a wrong place, it may cover normal blood branches supplying other important organs and cause severe problems like ischemic stroke. If the stent can be retrieved from a wrong location and redeployed in the desirable position, severe problems can be avoided. However, the current stents available for stent-assisted coiling or the current covered stents available can not be retrieved at all. Once these stents are deployed, they cannot be retrieved and redeployed even if the stents are misdeployed in an undesirable position. For a retrievable stent, it can be retrieved, readjusted and redeployed to the most desirable position, and in this way, it will greatly benefit the endovascular treatment of vascular diseases. Consequently, there is a need for the invention of a retrievable stent or a retrievable covered stent to facilitate the endovascular treatment of the above vascular diseases.

### BRIEF SUMMARY OF THE INVENTION

The present invention is related to a retrievable stent or covered stent which is particularly useful for treating intracranial sidewall aneurysms of blood vessels. The retrievable stent has a proximal retrieving structure and distal stent struts. The stent can be deployed across the neck of a sidewall aneurysm for assisting coil embolization or for neck obliteration in the case of a covered stent. If the retrievable stent is not deployed in the best location, the retrieving structure can be used to retrieve, readjust and redeploy the stent in the best position for treating the aneurysm. Examples of sidewall aneurysms where the retrievable stent may be implanted include, without limitation: the basilar artery, the middle cerebral, the internal carotid artery, the anterior communicating, and other arteries.

### BRIEF DESCRIPTION OF THE DRAWINGS OF THE INVENTION

Examples of embodiments of the invention will now be described with reference to the following drawings.

FIG. 1 shows the structure of the retrievable stent without a membrane. In FIG. A, the stent is fully expanded, whereas in FIG. B, the stent is compressed. When the stent is in the compressed state, the proximal end of the stent, namely the retrieving structure (113), will form a smaller profile than more distal segments of the stent. 101 indicates the small markers at the distal end of the stent, whereas 102 and 103 indicate the small markers at the proximal end of the stent.

FIG. 2 demonstrates the structure of the retrievable covered stent. In FIG. A, the stent is fully expanded, and in FIG. C, the stent is in a compressed state. When the stent is in the compressed state, the proximal end of the stent, namely the retrieving structure (113), will form a smaller profile than more distal segments of the stent. FIG. B indicates the piece of membrane or draft used for covering the stent.

FIG. 3 demonstrates the proximal retrieving structure of the retrievable stent which is a particular structure comprising a flexible circular metallic wire (111) with some markers (102 and 103) on it (A) or on the nearby most prominent stent struts (B, 112). Note, the proximal ends of the struts (112) of the retrievable structure form a slope or an incline plane rather than a horizontal or vertical plane. This particular structure enables the whole retrievable stent to be retrievable.

FIG. 4 indicates the process of the deployment of a retrievable stent in the technique of stent-assisting coil embolization for a sidewall aneurysm. 104 indicates a guidewire, 105 the catheter containing the retrievable stent, 106 the pushing structure for deploying the retrievable stent, 107 the vessel wall, 108 the catheter for coil embolization, and 109 the coils in the aneurysm cavity.

FIG. 5 illustrates the process of the deployment of a retrievable covered stent for the treatment of a sidewall aneurysm.

FIG. 6 shows the process of stent retrieval and redeployment. 110 indicates the hook for retrieving the stent, and 108 the catheter for containing the retrievable stent.

### DETAILED DESCRIPTION OF THE DRAWINGS OF THE INVENTION

Although the following discussion focuses on the treatment of vascular diseases, it is equally applicable to diseases in other locations or tracts. Although many types of vascular diseases can be treated with this retrievable stent or covered stent such as aneurysms, vascular defects and stenosis, the following discussion focuses on the treatment of sidewall intracranial aneurysms of blood vessels.

While this invention may be embodied in many different forms, there are described in detail herein specific embodiments of the invention. This description is an exemplification of the principles of the invention and is not intended to limit the invention to the particular embodiments illustrated.

As indicated above, the present invention is directed to a variety of embodiments.

Illustrated in FIG. 1 is an exemplary design of the retrievable stent in different states of expansion or compression. FIG. A demonstrates the retrievable stent in the expansion state with three markers (102 and 103) on the proximal end of the retrieving structure and two markers (101) on the distal end of the retrievable stent. These markers help visualization of the whole stent during and after deployment. The three markers (102 and 103) on the proximal end of the retrieving structure help delineate the proximal end of the retrieving structure. The two proximal markers (102) help decide the point of retrieval at the proximal retrieving structure for retrieval of the stent in case the stent is not deployed in the desirable location. The distal markers (101) at the distal end of the retrievable stent are useful for exact localization of the distal end of the stent in the blood vessels so that the sidewall aneurysm neck can be localized between the proximal marker (103) and the distal markers (101). When the stent is in the compressed state (FIG. B), the proximal end of the stent, namely the retrieving structure (113), will form a profile much smaller than the other more distal segments of the stent, and this small profile makes it easy to get into a catheter when the proximal retrieving structure is pulled into the catheter for retrieval. This is the mechanism to retrieve the whole stent. If the whole retrievable stent is not properly deployed in the desirable location, a hook can be used to capture the proximal retrieving structure between the two proximal markers (102) to retrieve and readjust the whole stent before re-deployment again. The stent used in this retrievable stent disclosed herein may be manufactured using any suitable known techniques to make a metal sheet into a stent or to braid or weld metallic wires into a stent. The material used for the retrievable stent can be of any type including steel and nitinol, and the retrievable can be closed-cell design or open-cell design.

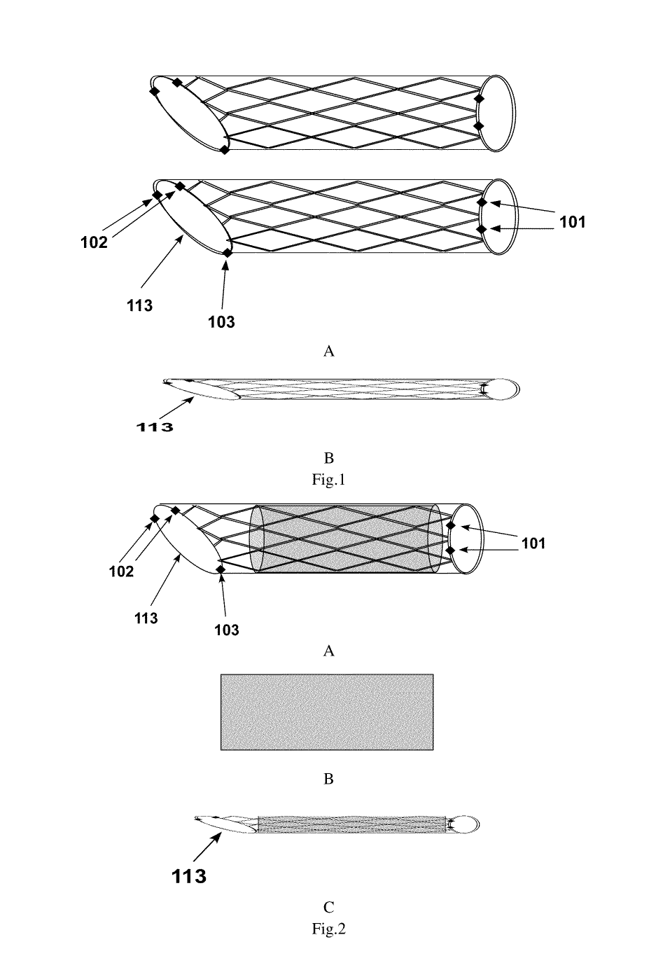

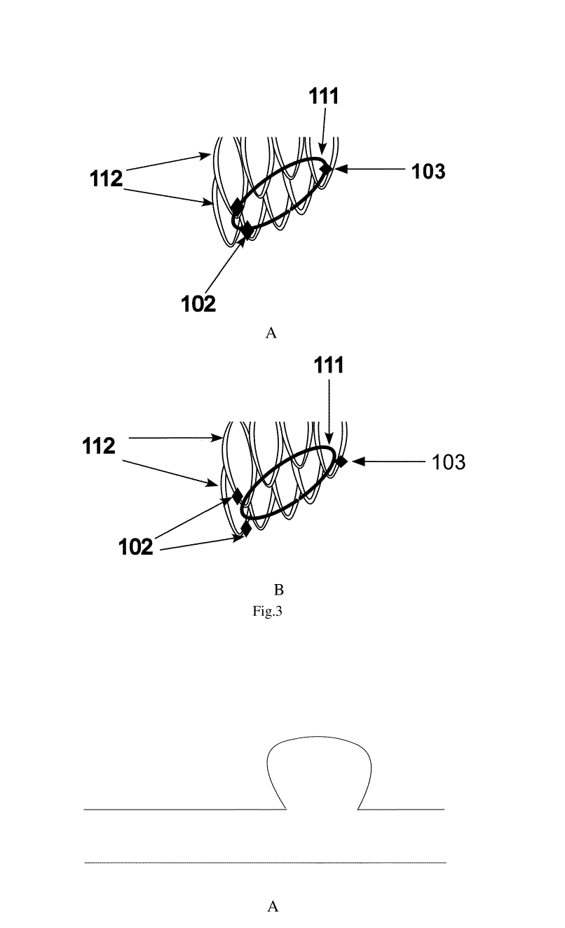

When the stent is covered with a piece of membrane and becomes a covered stent as demonstrated in FIG. 2, the proximal retrieving structure and the proximal and distal markers on the stent remain the same as in FIG. 1. In the case of a retrievable covered stent, the sidewall aneurysm neck is best covered by the membrane on the stent between the proximal marker (103) and the distal markers (101). If the aneurysm neck is not covered by the stent membrane, the covered stent can be retrieved, readjusted and re-deployed in the best location. The covering membrane or graft can be of any type including biomedical materials like polytetrafluoroethylene (PTFE) and other macromolecular materials. The graft or membrane can be stitched or glued onto the internal surface of the stent. The membrane used to cover the stent in this invention includes suitable polymer materials like polycarboxylic acids, cellulosic polymers, nylon, collagen, PTFE and expandable PTFE, polyethylene terephthalate and other medial materials. The stent used in this retrievable stent disclosed herein may be manufactured using any suitable known techniques to make a metal sheet into a stent or to braid or weld metallic wires into a stent. The material used for the retrievable stent can be of any type including steel and nitinol, and the retrievable can be closed-cell design or open-cell design.

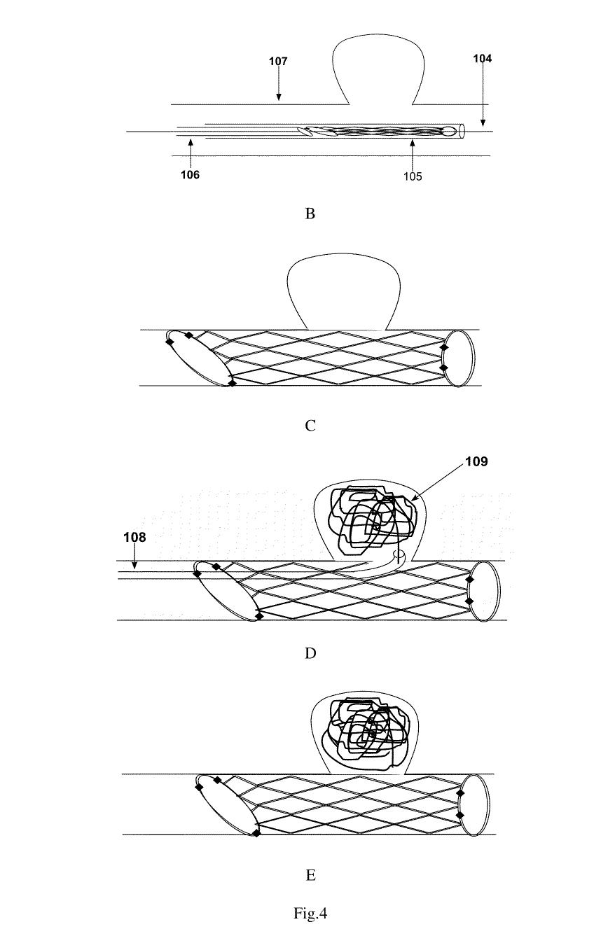

As shown in FIG. 3, the proximal retrieving structure is consisted of a flexible circular metallic wire (111) and three markers (102 and 103) on it (A) or on the nearby stent struts (B, 112). The flexible circular wire (111) goes through the proximal stent struts (112) whose ends are not on the same horizontal level but on a slope. This particular structure enables the whole stent to be retrieved. When the flexible metallic wire (111) is pulled by a retrieving hook which hooks up the circular wire (111) between the two markers (102), the struts (112) will gather toward the hook. If the whole stent is fully expanded and in full contact with vascular walls, this action of pulling the flexible metallic wire (111) will enable the proximal part of the stent to contract and gradually detach from the vessel wall. If the flexible wire (111) is pulled into a catheter, the struts (112) near the hook will get into the catheter first, and the struts farther away will follow into the catheter some time later. Because the end struts (112) of the proximal retrieving structure are not on the same horizontal or vertical level, the end struts will form a smaller profile enough to enter the catheter mouth easily. In this way, the struts of the proximal end of the retrieving stent will enter the catheter gradually and step by step, with some struts entering earlier while others later. Thus, the whole stent can be retrieved from the fully deployed state.

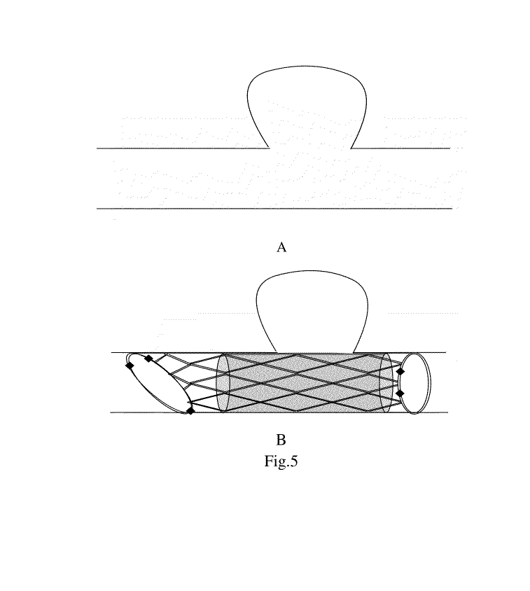

Referring to FIG. 4, the process of the deployment of the retrievable stent in the technique of stent-assisting coil embolization for treating a sidewall aneurysm is demonstrated. FIG. A shows a sidewall aneurysm. In FIG. B, a guidewire (104) is first introduced in the parent artery beyond the aneurysm neck, and then, the catheter (105) containing the retrievable stent is navigated along the guidewire to the aneurysm neck. After the retrievable stent is located in the right position, a pushing structure (106) is used to push the retrievable stent out to cover the aneurysm neck while withdrawing the catheter slowly until the stent is completely released as demonstrated in FIG. C. After the aneurysm neck is covered by the stent between the proximal marker (103) and the distal markers (101), a catheter 108 is introduced into the aneurysm cavity through the retrievable stent struts for coil embolization (FIG. D). After the aneurysm cavity is filled with coils (109), the catheter is withdrawn and the aneurysm is completely occluded by coils (FIG. E).

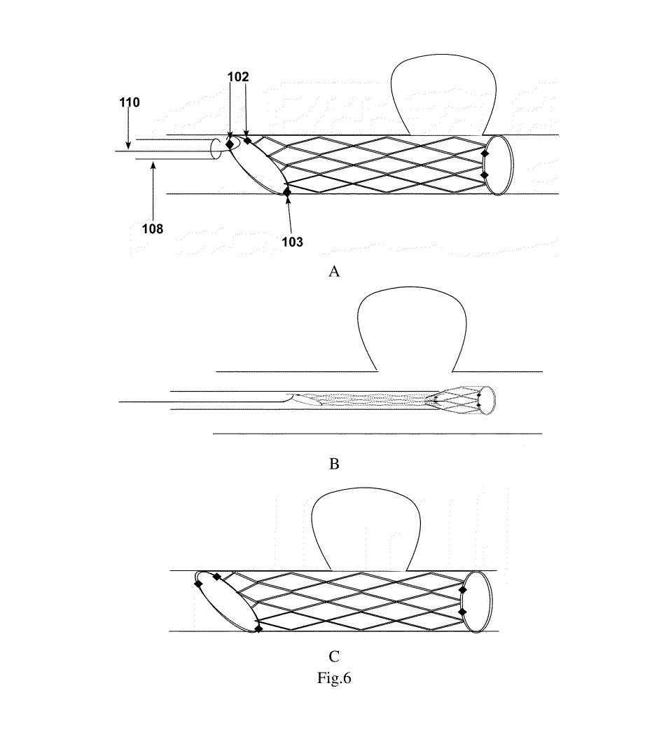

With reference to FIG. 5, a retrievable covered stent is delivered to cover the sidewall aneurysm neck (FIG. A) and once the covered stent is deployed in the required position (FIG. B), the aneurysm neck is occluded by the membrane on the stent, blood will no longer get into the aneurysm cavity, and thrombosis will be induced in the aneurysm cavity to completely obliterate the aneurysm from blood circulation.

Referring to FIG. 6, if the retrievable stent is not deployed in the required position (FIG. A), a catheter (108) with a hook (110) can be navigated to the stent, and the hook can capture the metallic wire of the proximal retrieving structure between the two markers (102) to withdraw the stent into the catheter. When the metallic wire is pulled, the proximal retrieving structure will contract and gradually detach from the vessel wall. The smaller profile of the proximal retrieving structure in the compressed state will facilitate easy entrance into the catheter. When most of the retrievable stent is located in the catheter and at the same time, the whole stent is detached from the vessel wall (FIG. B), the stent can be adjusted in position and redeployed in the required location to best cover the aneurysm neck (FIG. C).

What is claimed:

1. A retrievable stent which is used to treat intracranial sidewall aneurysms of vessels comprising:
a. a distal self-expandable or balloon-expandable stent or covered stent with some markers at the distal end;
b. a proximal retrieving structure connected to the distal self-expandable or balloon-expandable stent or covered stent for retrieving the whole stent;

2. The retrievable stent of claim 1, wherein the distal self-expandable or balloon-expandable stent or covered stent can be expanded and compressed with two different states of expansion and compression.

3. The retrievable stent of claim 2, wherein the distal self-expandable or balloon-expandable stent or covered stent has some markers at the distal end.

4. The retrievable covered stent of claim 2, wherein the graft material or membrane of the distal self-expandable or balloon-expandable covered stent is stitched or glued to cover the internal surface of the distal stent.

5. The retrievable stent of claim 2, wherein the distal self-expandable or balloon-expandable stent or covered stent can be made of shape-memory alloy like nitinol or steel or any other materials, and can be expanded by itself or by a balloon.

6. The retrievable stent of claim 1, wherein the proximal retrieving structure can be used to retrieve the whole stent.

7. The retrievable stent of claim 6, wherein the proximal end of the proximal retrieving structure forms a slope or an incline plane rather than a horizontal or vertical plane.

8. The retrievable stent of claim 6, wherein the proximal retrieving structure has a flexible circular metallic wire which connects or passes through the stent struts at the proximal end.

9. The retrievable stent of claim 6, wherein the flexible circular metallic wire of the proximal structure can be made of metal, alloy or any other strong materials for pulling and retrieving the whole stent.

10. The retrievable stent of claim 6, wherein the proximal retrieving structure has two or more markers on the circular metallic wire or on the nearby most prominent struts and struts opposite the most prominent struts.

11. The retrievable stent of claim 6, wherein the markers of the proximal retrieving structure can be used to delineate the proximal end and to localize the retrieval point.

12. A method for retrieving an endovascular stent or covered stent:
a. the retrievable stent or covered stent should have the stent struts at one end forming a slope or an incline plane rather than a horizontal or vertical plane even though the other end of the stent may be slope, horizontal or vertical;
b. when in the compressed state, the end of the struts forming a slope or an incline plane will constitute a smaller profile than the other segments of the stent;
c. a circular wire connects the stent struts at the end forming a slope for retrieval of the whole stent;
d. when the circular wire is captured by a hook and pulled toward the mouth of a catheter, the pulling of the wire will enable the proximal struts of the stent to compress and get into the catheter, and more distal struts will follow into the catheter until the whole stent enters the catheter gradually rather than all at the same time.
